(19) United States
(12) Patent Application Publication
(10) Pub. No.: US 2006/0030783 A1
(43) Pub. Date: Feb. 9, 2006

(54) Weight and body fat measurement device with temperature measuring capability

(76) Inventors: Ching Ching Tsai, Sindian City (TW); Hsueh Shun Lu, Bade City (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/016,089

(22) Filed: Dec. 17, 2004

(30) Foreign Application Priority Data
Aug. 8, 2004 (CN) 2004200881186

(51) Int. Cl. A61B 5/05; A61B 5/00

(52) U.S. Cl. 600/547; 600/549

(57) ABSTRACT

A measurement device includes a housing and a display unit mounted on a surface of the housing. A weight measurement unit and body fat measurement unit are disposed in the housing for measuring a load signal and a bioelectrical impedance signal. A temperature measurement unit is disposed in the housing for measuring a temperature signal. A controlling unit causes user-specific management information including a weight value, a body fat value and a temperature value to be transmitted to the display unit.

### BACKGROUND OF THE INVENTION

The invention relates to the field of measurement devices. More particularly, the invention relates to the field of weight or body fat measurement devices.

Weight or body fat control has become a modern obsession. A weighing instrument with body fat meter is an important tool used by many people in a program of weight and body fat control or management.

Furthermore, although several prior art scales have attempted to provide one or more of information to the user, there exists a need in the art for a scale that provides the user with several of user-specific health management information.

### SUMMARY OF THE INVENTION

An exemplary embodiment of the present invention overcomes the above-described problems by providing a measurement device that could store or display a user-specific management information at least including a temperature value and one of weight value and body fat value or combination thereof.

An exemplary embodiment of the invention provides a measurement device including a housing and a display unit mounted on a surface of the housing. A weight measurement unit is disposed in the housing for measuring a load signal and a temperature measurement unit is disposed in the housing for measuring a temperature signal. A controlling unit calculates a body weight value and a body temperature value from the load signal received from the weight measurement unit and the temperature signal received from the temperature measurement unit, and causes a user-specific management information including a weight value and a temperature value to be transmitted to the display unit.

Another exemplary embodiment of the invention provides a measurement device including a housing and a display unit mounted on a surface of the housing. A body fat measurement unit is disposed in the housing for measuring a bioelectrical impedance signal and a temperature measurement unit is disposed in the housing for measuring a temperature signal. A controlling unit calculates a body fat value and a body temperature value from the bioelectrical impedance signal received from the body fat measurement unit and the temperature signal received from the temperature measurement unit, and causes a user-specific management information including a body fat value and a temperature value to be transmitted to the display unit.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a top view of a measurement device according to an exemplary embodiment of the present invention;

FIG. 2 is a block diagram illustrating the components of a measurement device according to an exemplary embodiment of the present invention;

FIG. 3 is a block diagram illustrating the components of a measurement device according to an exemplary embodiment of the present invention; and

FIG. 4 illustrates a perspective view of a measurement device according to an exemplary embodiment of the present invention.

### DETAILED DESCRIPTION OF THE INVENTION

Various aspects of the system and method of the present invention will be described, and for purposes of explanation, specific configurations and details are set forth in order to provide a thorough understanding of the present invention. However, it will be apparent to one skilled in the art that the present invention may be practiced without these specific details. Furthermore, well known features have been omitted or simplified in order to prevent obscuring the present invention.

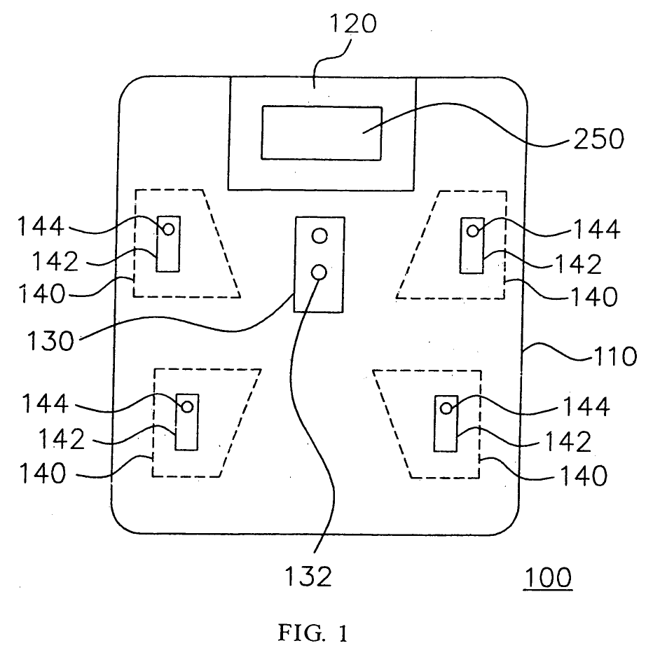

FIG. 1 illustrates the upper surface of measurement device such as digital scale 100, and the various components mounted on its housing 110, according to an exemplary embodiment of the present invention. A platform is disposed on the upper surface of the housing 110. Housing 110 may be constructed of metal or a reinforced plastic, such as glass or carbon-filled polymers. The platform substantially includes, for example, a display area 120 and sensing areas 140. A display unit 250 is mounted on the display area 120 and sensing devices 142, 144 and 310 are disposed on or under the sensing are a 140.

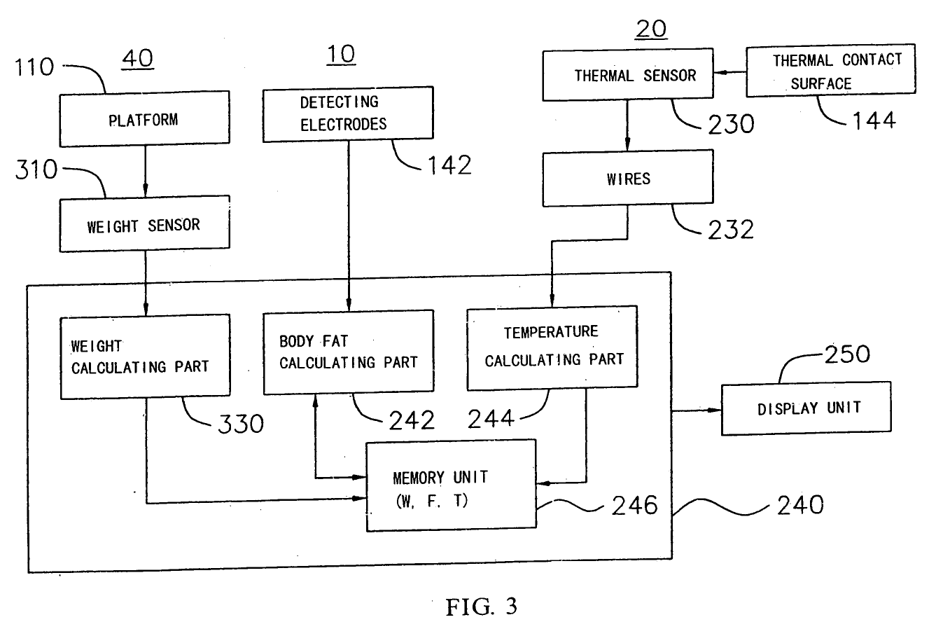

What are suitably disposed within housing 110 are a weight measurement unit 40 for measuring a load signal, a body fat measurement unit 10 for measuring a bioelectrical impedance signal and a temperature measurement unit 20 for measuring a body temperature signal, further depicted in FIG. 3.

In an exemplary embodiment of the present invention, the weight measurement unit 40 includes the platform and a weight sensor 310 disposed under the weighing platform for sensing the load signal. Typically, the weight sensor 310 could be a load sensing device including standard loadcell technology employed to measure weight. And a body weight value W is calculated by, for example, a weight calculating part 330 of a controlling unit 240 such as a central processing unit (CPU).

In this exemplary embodiment of the present invention, the body fat measurement unit 10 includes detecting electrodes 142 on the platform for measuring a bioelectrical impedance signal. Typically, in a body fat calculating part 242 of the controlling unit 240, the bioelectrical impedance signal is calculated by a four terminal electrode process. And the body fat value F is calculated by the body fat calculating part 242 based on the body weight value W and personal data such as sexuality and body height. The body fat value F could include the proportion of body fat, the weight of the body fat and the degree of obesity.

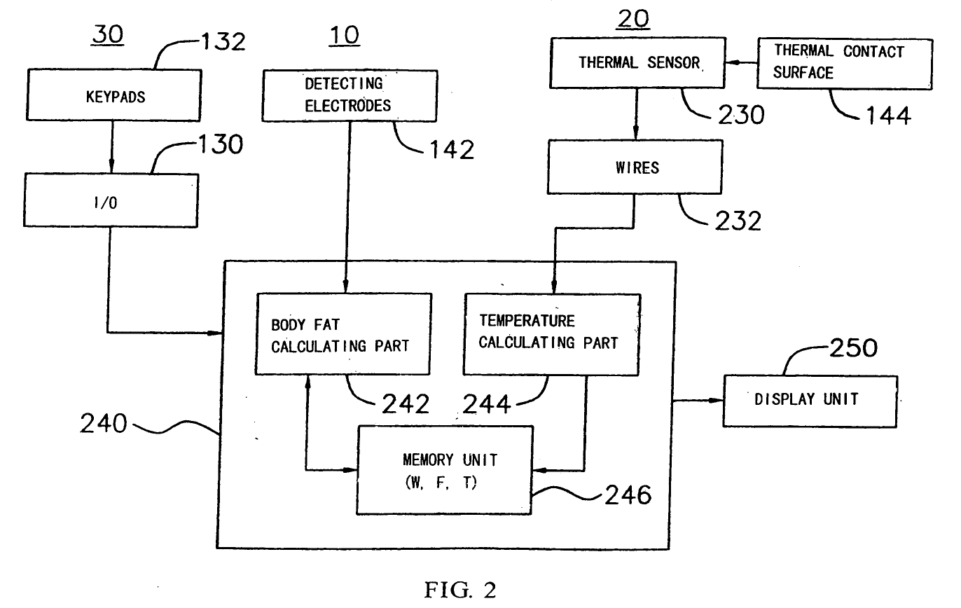

Referring to FIG. 1 and FIG. 2, in an exemplary embodiment of the present invention, the body weight value W and the personal physical data such as sexuality and body height could be obtained from an input unit 30 or memory unit 246 of the controlling unit 240. Typically, the input unit 30 could include keypads 132 and an input/output component (I/O) 130. Each of the keypads 132 is electrically coupled to the standard input/output component (I/O) 130, such as an analog to digital (A/D) converter. I/O 130 is coupled, for example, to CPU 240 directly or via common data bus. Thus, a user inputs information by actuating the keypads 132 which send appropriate signals to I/O device 130. I/O device 130 translates these signals into digital physical data which are sent to CPU 240.

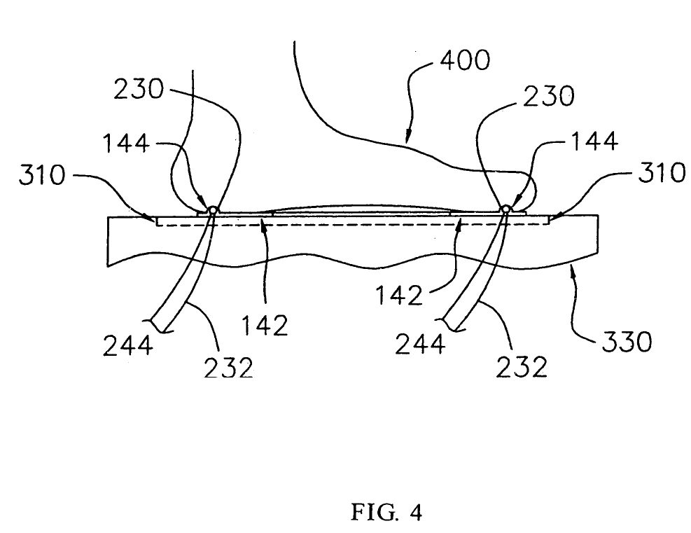

Referring to FIG. 1-FIG. 4, in an exemplary embodiment of the present invention, the temperature measurement unit 20 includes a thermal contact surface 144, a thermal sensor 230 and a set of wires 232. The thermal contact surface 144 is disposed on the upper surface of the housing 110 and could be located on the detecting electrodes. The thermal sensor 2230 is disposed within the thermal contact surface 144 for sensing the body temperature signal such as user's foot temperature signal. And the set of wires 232 are connected to the thermal sensor 230 for passing the body temperature signal to a body temperature calculating part 244 of the controlling unit 240.

Typically, the temperature measurement unit 20 could include an electronic thermometer or an infrared thermometer employed to measure temperature of user's body or foot 400. And a body temperature value T is calculated by, for example, a temperature calculating part 244 of the controlling unit 240. In an exemplary embodiment of the invention, the thermal contact surface 144 could include a collecting window made of a material transparent to heat radiation. By directly contacting the collecting window to an area such as the user's foot, the heat radiation emitted therefrom is collected. An infrared sensor could receive the heat radiation, which is then converted into the corresponding temperature signal transmitted to the controlling unit 240.

Thus, the controlling unit 240 could calculate the body temperature value T, the body weight value W or the body fat value F from the temperature signal received from the body fat measurement unit 10, the load signal received from the weight measurement unit 40 and the bioelectrical impedance signal received from the body fat measurement unit 10.

Therefore, the controlling unit 240 could cause a user-specific management information including the body weight value W, the body fat value F and the body temperature value T to be transmitted to a display unit 250 or stored in the memory unit 246 thereof.

Typically, CPU 240 could include a conventional microprocessor or a microcontroller. The user-specific control information is output to digital display 250 disposed on the display area 120, which is coupled to CPU 240 via a common data bus.

In addition to what mentioned above, typically, the inputted physical data could further include blood pressure or blood sugar, and the other user's body health information. Then, the physical data could be recorded in the memory unit 246 and merged into the user-specific control information.

Because the measurement device 100 could provide user-specific control information such as the foot temperature value and the weight or body fat value, it naturally provides a new home test that may help reduce the complications of diabetes. The monitors the skin temperature of feet to reduce complications including ulcers, fractures, and amputations.

The device could be used to provide objective information to patients so they would have an early warning sign of inflammation and tissue injury as part of a daily foot self-examination. Home temperature monitoring may be a useful adjunct to a comprehensive ulcer and amputation prevention program for patients at high risk for these complications.

While the invention has been described by way of example and in terms of the preferred embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A measurement device comprising:
a housing;
a weight measurement unit disposed in the housing for measuring a load signal;
a temperature measurement unit disposed in the housing for measuring a body temperature signal;
a display unit mounted on a surface of the housing; an
a controlling unit disposed in the housing and coupled to the weight measurement unit, the temperature measurement unit and the display unit;
wherein the controlling unit calculates a body weight value and a body temperature value from the load signal received from the weight measurement unit and the body temperature signal received from the temperature measurement unit;
wherein the controlling unit causes a user-specific management information including the body weight value and the body temperature value to be transmitted to the display unit.

2. The measurement device as recited in claim 1 wherein the weight measurement unit comprises:
a weighing platform disposed on an upper surface of the housing; and
a weight sensor disposed under the weighing platform for sensing the load signal.

3. The measurement device as recited in claim 1 wherein the temperature measurement unit is an electronic thermometer or an infrared thermometer.

4. The measurement device as recited in claim 1 wherein the temperature measurement unit comprises:
a thermal contact surface disposed on an upper surface of the housing;
a thermal sensor disposed within the thermal contact surface, for sensing the body temperature signal; and
a set of wires connected to the thermal sensor for passing the body temperature signal to the controlling unit.

5. The measurement device as recited in claim 1 wherein the controlling unit comprises a memory unit for storing the user-specific management information.

6. A measurement device comprising:
a housing;
a body fat measurement unit disposed in the housing for measuring a bioelectrical impedance signal;
a temperature measurement unit disposed in the housing for measuring a body temperature signal;
a display unit mounted on a surface of the housing; and
a controlling unit disposed in the housing and coupled to the body fat measurement unit, the temperature measurement unit and the display unit;
wherein the controlling unit calculates a body fat value and a body temperature value from the bioelectrical impedance signal received from the body fat measurement unit and the body temperature signal received from the temperature measurement unit;
wherein the controlling unit causes a user-specific management information including the body fat value and the body temperature value to be transmitted to the display unit.

7. The measurement device as recited in claim 6 wherein the temperature measurement unit comprises:
a thermal contact surface with a collecting window, disposed on an upper surface of the housing;
an infrared sensor disposed within the thermal contact surface, for receiving heat radiation from the collecting window, wherein the heat radiation is converted into the body temperature signal; and
a set of wires connected to the infrared sensor for passing the body temperature signal to the controlling unit.

8. The measurement device as recited in claim 6 further comprising an input unit disposed on the surface of the housing for inputting a physical data to the controlling unit, wherein the controlling unit calculates a body fat value from the bioelectrical impedance signal received from the body fat measurement unit and the physical data.

9. The measurement device as recited in claim 6 further comprising a weight measurement unit comprises:
a weighing platform disposed on an upper surface of the housing; and
a weight sensor disposed under the weighing platform for sensing a load signal;
wherein the controlling unit calculates a body fat value from the load signal and the bioelectrical impedance signal.
